(19) United States
(12) Patent Application Publication
(10) Pub. No.: US 2011/0236983 A1
(43) Pub. Date: Sep. 29, 2011

(54) SINGLE MOLECULE DETECTION AND SEQUENCING USING FLUORESCENCE LIFETIME IMAGING

(76) Inventors: Joseph Beechem, Eugene, OR (US); Theofilos Kotseroglou, Hillsborough, CA (US); William Michael Lafferty, Encinitas, CA (US); Mark F. Oldham, Emerald Hills, CA (US)

(21) Appl. No.: 12/980,817

(22) Filed: Dec. 29, 2010

Related U.S. Application Data
(60) Provisional application No. 61/290,734, filed on Dec. 29, 2009.
(60) Provisional application No. 61/296,624, filed on Jan. 20, 2010.

(51) Int. Cl. G01N 33/50; G01N 21/64; B82Y 99/00

(52) U.S. Cl. 436/94; 422/82.08; 977/774

(57) ABSTRACT

A nucleic acid detection system and method are provided, in which excitation energy is transmitted from a pulsed excitation source to a reaction site including a fluorescence resonance energy transfer (FRET)-based dye system to generate a fluorescent signal at the reaction site, the fluorescent signal is detected by a detector from the reaction site, and detection of the fluorescent signal is respectively blocked and permitted at the detector by a detector gate this is timed based on an emission start time of the transmitted excitation energy.

### CROSS-REFERENCE TO RELATED APPLICATIONS

This application claims the benefit of U.S. Provisional Application No. 61/290,734, filed Dec. 29, 2009, and U.S. Provisional Application No. 61/296,624, filed Jan. 20, 2010, both of which are hereby incorporated by reference herein in their entireties.

### FIELD

The present teachings relate to the fields of nucleic acid (e.g., DNA and all forms of modified DNA such as methylated DNA, and all forms of RNA, such as microRNA, non-coding RNA, etc.) detection and sequencing.

### INTRODUCTION

Fluorescence imaging techniques can utilize several different approaches to achieve contrast, including intensity, spectrum and lifetime. Fluorescence lifetime imaging (FLIM) is an imaging technique in which an image is produced based on the decay rate from a fluorescent sample. As a temporally resolved imaging modality, FLIM is relatively insensitive to local intensity variations. In some applications, FLIM utilizes ultrafast laser technology or laser sources attenuated with a high frequency modulation as an excitation source.

Recently, single molecule nucleic acid sequencing has been introduced in which a fluorescently labeled nucleotide polyphosphate incorporates into a growing nucleotide strand at an active site complementary to a target nucleic acid molecule for which sequencing is desired. In one methodology, fluorescent emission signals resulting from fluorescence resonance energy transfer (FRET) between a donor, such as a semiconductor nanocrystal, at the site of the next base to be called in the target nucleic acid molecule and the fluorescently labeled nucleotide polyphosphate can be detected and converted into a base call to ultimately reveal the target nucleic acid sequence. Reference is made to International Publication No. WO/2010/111674, entitled “Methods and Apparatus for Single Molecule Sequencing Using Energy Transfer Detection,” which is incorporated by reference herein in its entirety. In a FRET system, an excited donor moiety subsequently transfers its energy to an acceptor moiety only in close vicinity to the donor moiety. A fluorescent signal can then be detected from the acceptor moiety, which signal is generally spectrally separated from the donor moiety.

Single molecule nucleic acid sequencing is very sensitive to optical noise that is generated from the excitation source and due to limitations resulting from collection optics, for example, background fluorescence from index matching oil, and glue used in objectives. Sometimes the noise cannot be distinguished from a signal generated by a sequencing reaction, for example, a signal generated from the incorporation of a dye-labeled nucleotide by an enzymatic reaction and a corresponding emission of fluorescence from a directly excited dye-labeled nucleotide not bound to the DNA polymerase. Since the emission spectrum of the directly excited dye-nucleotides are often identical to the dye-nucleotides bound to the DNA polymerase (that generate the true sequencing information signal), spectral methods are not well suited for separating these two emission signals. However, the dye-labeled nucleotides bound to the DNA polymerase (the true sequencing signal component) will have a distinct fluorescence lifetime that matches very closely the fluorescence lifetime of the donor-emitting component and hence is resolvable from the directly excited dye-labeled nucleotides.

Furthermore, if FRET is involved, where an excited donor subsequently transfers its energy to an acceptor only in close vicinity, the efficiency of this process is less for acceptors with less overlap with the donor, for example, redder absorbing acceptors for donors emitting in the blue through yellow region of the spectrum. The further the absorption spectrum of the acceptor is from the emission spectrum of the donor, the fewer photons are generated by the acceptor due to FRET. Thus, very weak acceptor signals are inherent in FRET sequencing in these occasions.

Therefore, a system and method of increasing the signal-to-noise ratio of such a process by decreasing the noise and/or increasing the signal would be desirable. In particular, a system and method of increasing the signal-to-noise ratio without the need for additional filtering devices to filter the noise or amplifiers to increase the signal would be desirable. Additional hardware or additional filtering techniques generally require greater expense or additional time to achieve accurate detection of the acceptor signal. Thus, while the signal-to-noise ratio may increase, the amplification of the detected signal or the filtering of unwanted noise can result in additional time or expense in single molecule nucleic acid sequencing. Accordingly, a need exists for faster, less expensive, more reliable, single molecule detection and sequencing systems and methods.

### SUMMARY

The present teachings may solve one or more of the above-mentioned problems. Other features and/or advantages may become apparent from the description which follows.

Additional objects and advantages may be set forth in part in the description which follows, and in part will be obvious from the description, or may be learned by practice of the present teachings. Those objects and advantages will be realized and attained by means of the elements and combinations particularly pointed out in the appended claims.

It is to be understood that both the foregoing general description and the following detailed description are exemplary and explanatory only and are not restrictive of the present teachings or claims.

In accordance with at least one exemplary embodiment, the present teachings contemplate a nucleic acid detection system including a pulsed excitation source, a detector, and a detector gate. The pulsed excitation source transmits excitation energy to a reaction site including a fluorescence resonance energy transfer (FRET)-based dye system to generate a fluorescent signal at the reaction site. The detector is configured to detect the fluorescent signal from the reaction site. The detector gate is configured to respectively block and permit detection of the fluorescent signal at the detector, the detector gate being timed based on an emission start time of the transmitted excitation energy.

In accordance with at least one exemplary embodiment, the present teachings contemplate a method of detecting a nucleic acid molecule sequence. The method includes reacting at a reaction site a nucleic acid molecule with a fluorescence resonance energy transfer (FRET)-based dye system. The method further includes turning on an excitation source and transmitting excitation energy to the FRET-based dye system to generate fluorescent emissions at the reaction site. The method includes preventing detection, by a detector gate, of the fluorescent emissions at a detector after an emission start time of the transmitted excitation energy. The method further turns off the excitation source, and permitting detection of the fluorescent emissions after a first predetermined amount of time from the emission start time has elapsed. The method detects the fluorescent emissions with the detector to form a detected signal, and determines a character or sequence of the DNA molecule based on the detected signal.

In accordance with at least one exemplary embodiment, the present teachings contemplate a method of detecting a nucleic acid molecule sequence that includes reacting at a reaction site a nucleic acid molecule with a fluorescence resonance energy transfer (FRET)-based dye system. The method further includes transmitting excitation energy to the reaction site to excite the FRET-based dye system to generate fluorescent emissions at the reaction site. The method also includes syncing timing of the excitation energy transmission with detection of the fluorescent emissions by a detector and detecting the fluorescence at the detector after a predetermined delay time from an emission start time of the transmitted excitation energy.

### BRIEF DESCRIPTION OF THE DRAWINGS

The accompanying drawings, which are incorporated in and constitute a part of this specification, illustrate various exemplary embodiments of the present teachings and together with the description, serve to explain certain principles. In the drawings:

FIG. 1 is a schematic flow diagram showing time-gated fluorescence lifetime imaging detection according to various exemplary embodiments of the present teachings;

FIG. 2 is a schematic representation of an exemplary embodiment of a nucleic acid detection system according to the present teachings;

FIG. 3 is a schematic side view of another exemplary embodiment of a nucleic acid detection system according to the present teachings;

FIG. 4 is a graph showing the fluorescence lifetimes of a number of different FRET-based dye systems utilizing quantum dots as the donor, showing the effect of dyes per dot on fluorescence intensity decay, and showing FRET dye configurations that can be used according to various embodiments of the present teachings;

FIG. 5 is a graph showing a curve of the rise time and decay time of an excitation beam, a curve of the fluorescence decay of a FRET-based dye system utilizing a quantum dot donor, and a curve showing the closed and open timing of a detector gate, in accordance with various exemplary embodiments of the present teachings;

FIG. 6 is a table showing the fluorescent lifetime of various dyes, which may be used as part of a FRET-based dye system (e.g., as acceptors) in accordance with various embodiments of the present teachings;

FIGS. 7A and 7B respectively show images of fluorescent emissions at a donor (D) and two acceptor (A1, A2) fluorescent emission channels without a time-gated detection system and with a time-gated detection system in accordance with various embodiments of the present teachings; and

FIG. 8 is a graph showing average intensities of fluorescent emissions observed over time in an acceptor channel and a donor channel in accordance with various embodiments of the present teachings.

### DETAILED DESCRIPTION

Reference will now be made in detail to various exemplary embodiments, some of which are illustrated in the accompanying drawings. Wherever possible, the same reference numbers will be used throughout the drawings to refer to the same or like parts.

To facilitate an understanding of the present teachings, the following definitions are provided. It is to be understood that, in general, terms not otherwise defined are to be given their ordinary meanings or meanings as generally accepted in the art.

As used herein, the term “detector gate” and variations thereof as used herein can include a variety of mechanisms or techniques that permit or limit detection of a signal by the detector. Detector gates can include optical-based approaches, such as, for example, electronic shutters or microchannel plates, or electronics-based approaches, such as, for example, timing circuitry used to turn pixel detection ability on and off. Detector gates, as used herein, can also include mechanisms for intensifying signals, as with the aforementioned microchannel plates.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. Thus, for example, reference to “a sample” can include two or more different samples. As used herein, the term “include” and its grammatical variants are intended to be non-limiting, such that recitation of items in a list is not to the exclusion of other like items that can be substituted or added to the listed items.

Various exemplary embodiments in accordance with the present teachings contemplate a system and method of increasing the signal-to-noise ratio during single molecule nucleic acid detection by minimizing noise, including, e.g., from fluorescent emissions coming from sources other than the fluorescent emissions generated through fluorescent resonance energy transfer of an acceptor dye, detected by the detector, particularly without the use of additional filtering devices and/or other optics structures.

In various exemplary embodiments, systems and methods for single molecule nucleic acid detection can utilize natural signal filtering by the use of specific sequencing reagents (e.g., dyes) having long fluorescent lifetimes and time-delayed detection mechanisms to naturally filter noise using the inherent sequencing reaction properties, as opposed to, for example, utilizing other optics structures and/or signal software processing techniques to distinguish noise from desired signals. Limiting the amount of background noise that is detected can provide more accurate detection of the fluorescent signal resulting from nucleotide incorporation and faster and less expensive sequencing due to more accurate detection of the fluorescent signal to be analyzed for sequencing analysis.

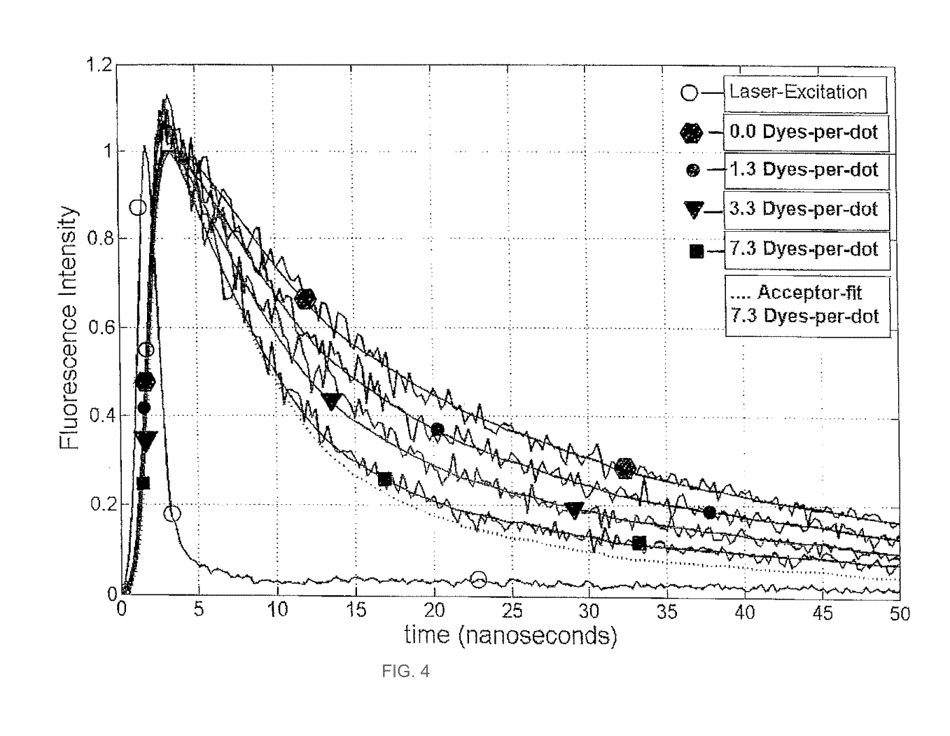

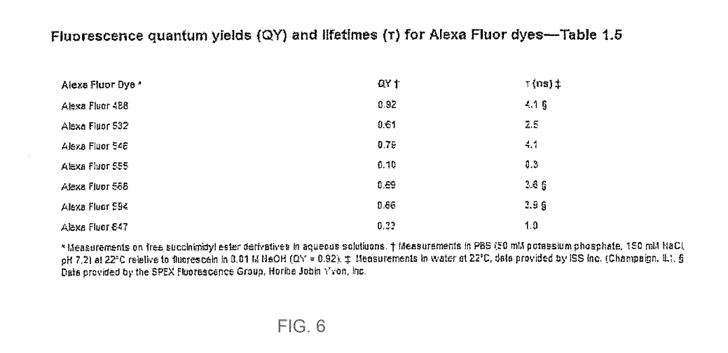

According to various exemplary embodiments of the present teachings, a FLIM detection technique is used to enhance signal quality for detection of a single molecule of nucleic acid, and for sequencing the same. According to various embodiments, a FRET-based dye system is used in conjunction with a FLIM detection system in a single molecule nucleic acid detection system, for example, in a single molecule detection or sequencing system. The system can include a pulsed excitation source, a detector gate, and a FRET-based dye system that uses a donor that exhibits fluorescence decay on the order of tens of nanoseconds to microseconds. In an exemplary embodiment, the FRET-based dye system can comprise one or more quantum dot donors, such as, for example, quantum dot donors that have fluorescent lifetimes very distinct from the acceptor-dyes utilized. For example, quantum-dots made with cadmium-selenide (CdSe) cores and zinc-sulfide (ZnS) shells can have fluorescence lifetimes that are much longer (10's to 100's of-nanoseconds; see FIG. 4, for example) than some fluorescence dyes typically used as acceptors (see FIG. 6, for example). Altering the method/type of layering and types of materials used in the core and shell can affect the magnitude of the fluorescence lifetimes of the donor quantum dots. In another exemplary embodiment, the FRET-based dye system can include various long fluorescence lifetime decay dyes as donors, including but not limited to, for example, lanthanides, acridines, and/or rutheniums. While quantum dots, lanthanides, acridines, and rutheniums are disclosed herein as exemplary donors, any of a variety of materials that cause relatively long (10 ns+) fluorescent decay times may be used as a donor in a FRET-based dye system in accordance with exemplary embodiments of the present teachings. The one or more dyes may be different fluorophores, or may be FRET pairs consisting of two or more different fluorophores. Both the donor and acceptor (e.g., quantum-dot/dye) elements may comprise not just a single donor/acceptor pair, but can comprise multiple-component FRET systems (e.g, multiple donors and/or acceptors). For instance, the acceptor dye could be composed of two different dyes, the first acceptor dye being the primary acceptor of the emission of the donor while the second acceptor dye is the primary acceptor of emission from the first acceptor dyes emission. In this manner, the acceptor emission can be shifted to longer wavelengths while maintaining higher net FRET efficiency. Similarly, combinations of multiple donor-types (e.g., multiple quantum-dot (Qdot) donors, or quantum-dot-dye-labeled donors) may be used to obtain more flexibility in the range of excitation wavelengths for sequencing DNA. In various exemplary embodiments, the FRET-based dye system can include a fluorescent dye, such as, for example, organic dyes, as an acceptor (see, e.g., FIG. 6) or any of all dye classes contained in Handbook of Fluorescent Probes and Research Chemicals, R. P. Haugland, 9^{th} ed., Molecular Probes Inc., Eugene, Oreg. (1996). Acceptor dye-nucleotide systems can also be constructed wherein the acceptor nucleotide is part of a nanoparticle (e.g., microsphere, quantum-dot, or streptavidin). The nanoparticle can be attached to the nucleotide at the terminal phosphate position of the nucleotide and released upon successful incorporation of the nucleotide base into the DNA.

In accordance with various exemplary embodiments, a FRET-based dye system can include a donor tethered to a nucleic acid polymerase molecule and an acceptor that labels a nucleotide (dye-labeled nucleotide). Upon excitation of the donor, such as, for example, via an excitation source, the donor fluoresces and energy from the donor is transmitted to an acceptor dye attached to a nucleotide in sufficiently close proximity to the donor, whereby the acceptor consequently emits a fluorescent signal as a result of the FRET. Since FRET can occur only when an acceptor is in sufficiently close proximity to the donor, only an acceptor dye on a nucleotide incorporated, or transiently bound, at the site of the base in a target nucleic acid molecule at which the polymerase is located will fluoresce via FRET. Utilizing a FRET-based dye system, therefore, allows for the primary detection of the fluorescent signal from the acceptor of the incorporated, or transiently bound, nucleotide. For further explanation of FRET-based techniques that can be utilized for single molecule sequencing, reference is made to International Publication Nos. WO/2010/111674, entitled “Methods and Apparatus for Single Molecule Sequencing Using Energy Transfer Detection,” and WO/2010/141390, entitled “Nucleotide Transient Binding for Sequencing Methods”, both of which are incorporated by reference herein in their entireties. Thus, autofluorescence caused by an excitation source or other non-FRET-based fluorescence emissions can be distinguished (e.g., removed) from the detection of the acceptor fluorescent signal, enabling enhanced detection and sequencing of single target nucleic acid molecules.

According to various exemplary embodiments, a nucleic acid detection system can include an excitation source, a detector, a detector gate, and a FRET dye system.

The detector can be configured to receive a fluorescent signal from a reaction site. The gain of the detector gate can be configured to rapidly increase and decrease and thus respectively allow passage of the amplified fluorescent signal to the detector and block passage of the un-amplified fluorescent signal to the detector. The FRET-based dye system can be configured to be excited by the excitation source and generate the fluorescent signal at the reaction site. In some embodiments, the FRET-based dye system can comprise at least four different FRET acceptor dyes, one for each nucleotide. However, the systems and methods of the present teachings can include any number of dyes, for example, from 1 to more than 1, for example, 2 or 5 dye systems also are contemplated.

According to various embodiments, the FRET-based dye system can include quantum dot donors, including but not limited to quantum dot nanocrystals, for example, of the S-dot-type. Examples of suitable types of Q-dots, and methods for making the same, that can be utilized in conjunction with the system and methods of the present teachings include, but are not limited to, for example, those disclosed in U.S. Patent Pub. No. 2010/0035268, entitled “Materials and Methods for Single Molecule Nucleic Acid Sequencing,” International Publication No. WO/2010/002939, entitled “Methods for Real Time Single Molecule Sequencing,” and U.S. Patent Pub. No. 2010/0255487, entitled “Methods and Apparatus for Single Molecule Sequencing Using Energy Transfer Detection”, the entire contents of all of which are incorporated by reference herein. In some exemplary embodiments, the detector can comprise one or more charge-coupled device (CCD) cameras or complementary metal oxide semiconductor (CMOS) cameras, or direct on-semiconductor (e.g., CMOS) chip detection. In some embodiments, the detector gate can comprise an intensifier/gate or image intensifier. In some embodiments, the system can further comprise a reaction site and a direct or indirect binding or reaction of a quantum dot, a nucleic acid (e.g., DNA) molecule, a polymerase, and a dye-labeled dNTP (dye-labeled nucleotide) at the reaction site.

According to various exemplary embodiments, a method of detecting a nucleic acid molecule is provided, and includes interactions of a dye-labeled nucleotide (such as, for example, a deoxyribonucleotide triphosphate (dNTP)) molecule with a FRET donor, such as, for example, a quantum dot or a long fluorescent lifetime dye. The interactions of the dye-labeled nucleotide molecule with a quantum dot or fluorescent lifetime dye can be considered to be a FRET-based dye system. The method can include exciting the FRET donor with an excitation source, configuring a detector gate to be in a low or non-amplified configuration to prevent or minimize detection of, for example, the excitation beam, autofluorescence from optical elements, and/or background from unincorporated, dye-labeled nucleotides which are not directly or indirectly bound or reacting with a quantum dot from reaching a detector. The method can further include turning off the excitation source, configuring a detection gate to be in an amplifying configuration after turning off the excitation source, and detecting the emission from an acceptor dye of a dye-labeled nucleotide incorporated or transiently bound at the reaction site. In an amplified configuration of the gate, the amplified emission can pass through the gate and reach the detector. In alternative embodiments, the method can comprise exciting the FRET donor via an excitation source, configuring an optoacoustic device which can modulate one of the excitation beam and/or the emission beam to prevent or minimize the excitation beam, or autofluorescence from optical elements, or background from bye-labeled nucleotides which are not directly or indirectly bound or reacting with the FRET donor from reaching a detector, modulating or turning off the excitation source, configuring a detection gate to be in an amplifying configuration after modulating or turning off the excitation source, and detecting the emission of a FRET acceptor with the detector to form a detected signal. In some embodiments, the detecting can comprise forming a detected signal, and the method can further comprise determining a base or sequence of the nucleic acid molecule based on the detected signal.

In some embodiments, a method is provided that comprises reacting the nucleic acid molecule with four different FRET-based dye systems. In some embodiments, the excitation source can comprise a pulsed or modulated laser source and the method can comprise repeatedly pulsing the pulsed or modulated laser source between an on configuration wherein the pulsed laser source generates an excitation beam that follows an optical path to a reaction site at which a nucleic acid molecule to be detected is located, and an off configuration wherein the pulsed laser source is turned off and does not generate an excitation beam, or is modulated, potentially to an alternate optical path, which does not intersect the reaction site such that the excitation beam does not excite the reaction site. In some embodiments, the method can further comprise intensifying the emission beam while detecting the emission beam with the detector. In some embodiments, the method can comprise sequencing the nucleic acid molecule.

In some embodiments, a system is provided which is configured to minimize the loss of resolution that can result from using an image intensifier. Such a system can comprise generating an image on an image intensifier, and reimaging the image from the output of the image intensifier onto a camera, where the size of the image on the image intensifier is larger (higher magnification) than the image generated on the camera.

According to various embodiments of the present teachings, a FRET-based dye system that uses quantum dots as FRET donors, can exhibit fluorescence decay on the order of many tens of nanoseconds, for example at least 20 ns, for example at least 40 ns, for example at least 60 ns, for example at least 75 ns, or for example, about 80 ns or longer. A FRET-based dye system that uses as FRET donors long fluorescent lifetime dyes, such as, for example, lanthanides, acridines and/or rutheniums, can exhibit fluorescence decay on the order of several microseconds up to several milliseconds. For many other types of dyes that may be used as FRET donors, on the other hand, typical fluorescence lifetimes are less than about 10 nanoseconds. Thus, FRET-based dye systems with donors having long lifetime fluorescent decay in conjunction with FLIM detection techniques can allow for longer periods of fluorescent detection than other types of donor materials. Longer periods of fluorescent detection are desirable because the detection of fluorescent signals emitted from the acceptor in the FRET system can be delayed to avoid detection of background noise from sources having shorter fluorescent lifetimes. Thus, background noise that has shorter fluorescent decay lifetimes than that of the fluorescent signals from the acceptor can be excluded from detection, thereby ultimately enhancing the signal-to-noise ratio of the detected signal and improving the accuracy of the detection and sequencing in a single molecule detection system.

According to various embodiments of the present teachings, a time-gated fluorescence detection system and method are provided. The system and method can be used to increase the ratio of signal-to-noise in fluorescence resonance energy transfer-based (FRET-based) single nucleic acid molecule sequencing. In some embodiments, the imaging-based system is nanosecond-gated and uses fluorescence lifetime imaging techniques and quantum dots (Q dots) to extend the fluorescence lifetime of FRET dyes, dye complexes or dye systems and subsequently detect fluorescent emission signals after the gate is opened to allow passage of the emission signals from the acceptor sites. In other embodiments that utilize long fluorescent lifetime dyes in lieu of Q-dots, the time gates that are used can be longer than nanoseconds, for example, from microseconds up to milliseconds. Those having ordinary skill in the art would understand based on the present teachings how to select appropriate excitation frequencies, time gates, etc. based on the type of FRET compounds used, to minimize or exclude detecting of autofluorescence and other emissions of relatively shorter fluorescence lifetimes.

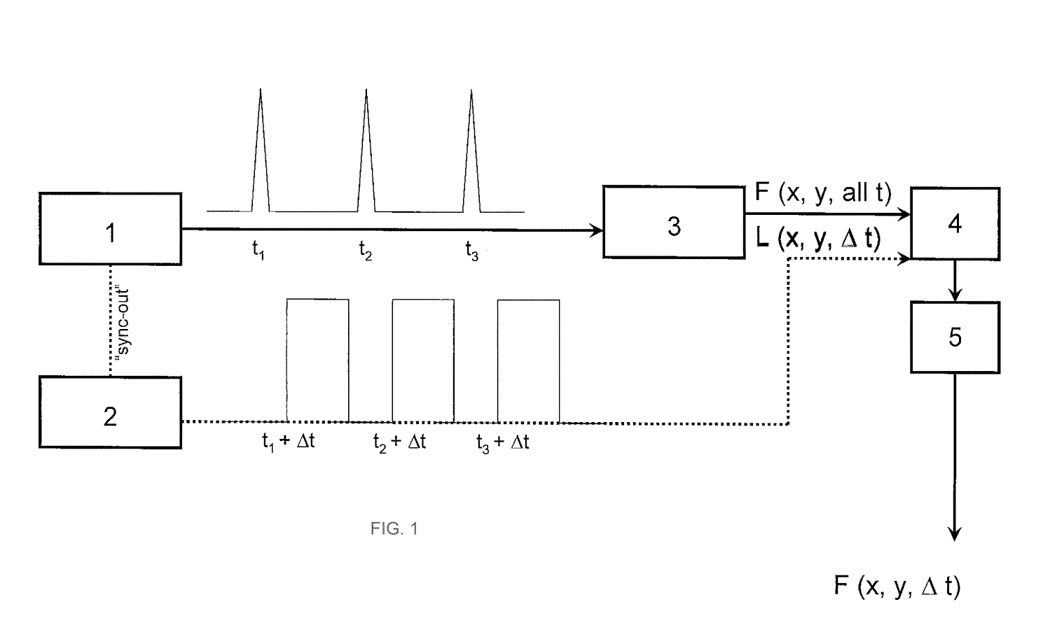

With reference to FIG. 1, an exemplary embodiment of a logic flow diagram for achieving a time-gated detection scheme for single nucleic acid molecule detection utilizing a FRET-based dye system and FLIM detection is depicted. As represented schematically in FIG. 1, energy is transmitted from an excitation source 1 to one or more reaction sites of a sample 3 containing one or more single nucleic acid molecules to be detected and/or sequenced. At the one or more reaction sites, a FRET donor (e.g., a quantum dot or long fluorescent lifetime dye) is tethered to a polymerase molecule that is positioned at the site of the base for which the next nucleotide incorporation (or transient binding when a transient binding sequencing reaction is implemented) event will take place. Excitation of the donor causes emission of fluorescence from the donor and a transfer of energy via FRET to an acceptor in the form of a dye labeling a nucleotide incorporated at the site of the next base. The dye labeling the incorporated nucleotide then emits its own fluorescent signal. Each of the four nucleotides can be terminally labeled with four different fluorescent dyes that act as the acceptors in the FRET-based dye system, and the dye-labeled nucleotides are added to start the sequencing of the nucleic acid molecule at a reaction site in the sample 3. Spectral resolution of the emitted fluorescence can occur at the level of component 3 or component 4 (or a combination of 3 and 4) and can comprise filter-based or diffractive-element-based (e.g., gratings) elements for spectral resolution. Multiples of components 4 and 5 can be utilized (working as pairs), each observing spectrally distinct emissions from the sample 3.

Using fluorescence lifetime imaging techniques and the duration of emission of the fluorescent signal from the acceptor, a digital delay/gate generator (detector gate) 2 can be used with, for example, a gating image intensifier 4 to block or permit detection of the fluorescent signal at a detector 5. In particular, the detector gate 2 may respectively cause detection of fluorescent signals to be blocked at the detector 5 for a predetermined length of time and then may allow detection of fluorescent signals at the detector 5. The detector gate 2 may be any mechanism that permits or blocks detection of the fluorescent signal by the detector 5 and may be embodied as an electronic shutter mechanism, an electric charge to permit or block passage of a fluorescent signal to the detector 5 or may be embodied as part of the detector 5 itself. For example, when incorporated as part of the detector 5, the detector gate 2 is the implementation of the timing of the detection by the detector 5. In this case, the sensitivity of the detector 5 is electronically biased to zero during the pulsed emission beam and then the detector 5 is turned back on at the appropriate time (e.g., pixels of the detector our turned on or off based on the detector gate 2 timing).

The detector gate 2 blocks detection of the fluorescent signal at the detector 5 for a length of time that permits background noise, the signals of which are emitted for time durations less than that of the fluorescent signal emitted from the FRET acceptor dye, to significantly dissipate. Generally, noise from directly-excited acceptors, emissions from other substances in the system (e.g., fluids, optical glues, plastic structures, etc.), and/or emission from the excitation source, have fluorescence lifetimes of less than 5 ns, while the fluorescent signals emitted from the acceptor moiety when using quantum dots, lanthanides, acridines, rutheniums, or other long fluorescence lifetime donors have much longer fluorescence lifetimes of, for example, at least 10 ns or more.

Thus, in order to limit or prevent the detection of outside noise, the detector gate 2 may be configured to block or permit detection of the fluorescent signal emitted by the acceptor, as will be discussed further below. The detector gate 2 initially blocks or otherwise prevents the detection of autofluorescence of various components of the detection system after a period of excitation. For example, after a period of about 10-15 ns, or other first predetermined amount of time, as will be discussed further below, the detector gate 2 allows the detection at the detector 5 of the fluorescent signal from the acceptor. As shown in FIG. 1, the timing of the detection may be synced with the pulsed or modulated excitation source 1 by operating the excitation source 1 and the detector gate 2 in sync with each other. By “in sync” what is meant is that digital delay/gate 2 is synchronous with, that is, in-phase with, excitation source 1.

Operatively connected to the digital delay is a detection system 3/4/5, which can take various configurations depending on the overall detection system used, examples of which will be explained further below. For example, in a system based on microscopy, element 3 can include an optical microscope to image the reacted sample, element 4 can be a gating image intensifier (e.g., a microchannel plate), and element 5 can be a detector that receives fluorescent signals from element 4, such as, for example, a CCD camera. In an exemplary operation of such an embodiment, therefore, laser pulses at 1 (less than the lifetime of the donor fluorescence) are input (shown by solid line in FIG. 1) to the optical microscope 3. The synchronous-out function on the laser (which can alternatively be replaced with a beam-split fraction of the pulsed laser beam) generates a delayed logic pulse 2 (shown by the dashed line in FIG. 1) relative to the timing of the laser pulse going into the microscope. The focused output light from the microscope unit 3 impinges on the gated-intensifier 4 which selects for the delayed light relative to the excitation pulses. Only the delayed light is focused and imaged by the imaging detector 5.

In another exemplary system based on a semiconductor (CMOS) chip, for example, element 3 can have a sample chamber integrated with a CMOS detector that includes a time delay detection mechanism 4 and fluorescent signal readout via element 5, such as, for example, a digitized voltage corresponding to pixels (which in an exemplary embodiment can in turn correspond to individual sequencing reaction sites within the sample chamber). In an exemplary operation of such an embodiment, therefore, laser pulses 1 (less than the lifetime of the donor fluorescence) are input (shown by solid line in FIG. 1) to the sample 3. The synchronous-out function on the laser (or replaced with a beam-split fraction of the pulsed laser beam) generates a delayed logic pulse 2 (shown by dashed line in FIG. 1) relative to the timing of the laser pulse 1. The output light from 3 impinges on a CMOS detector which is controlled by the logic from 2 to either be in an “on” state or “off” state to either detect or not detect the light emitted from 3. Only the detected light is then digitized to a voltage at 5. In another exemplary embodiment, an electronic shutter can be used in conjunction with the detector gate logic 2 to prevent emissions (e.g, from the excitation source, donor, non-FRETing acceptors, other system substances, etc.) from reaching the detector 4. The electronic shutter can be closed during the excitation and opened after the excitation source is turned off, in a manner consistent with the present teachings.

As further shown in FIG. 1, tₙ is the nth light pulse centered at time t. tₙ+Δt is the nth logic pulse with 100% “on” at t+Δt. At can be adjusted as desired depending, for example, on the various fluorescent lifetimes of the FRET acceptors and of other components of the system having emissions that are desired to be excluded. F(x, y, all t) is the fluorescent image at spatial coordinates (x, y) and all time. L (x, y, Δt) is the logic pulse used in gating the image intensifier/image detector. F (x, y, Δt) is the fluorescent image at spatial coordinates (x, y) delayed in time Δt from a pulse from excitation source 1. As can be seen, in FIG. 1, and mentioned above, the path of light is shown by the solid line arrows and the path of logic is shown by the dashed line and dashed line arrow.

Excitation pulses or modulations that are less than the lifetime of the donor fluorescence are thus input to the sample 3, for example, via an optical microscope or other CMOS chip-based sample chamber. In some embodiments, the synchronous-out function on the excitation source generates a delayed logic pulse relative to the timing of the excitation pulse/modulation going into the detector (e.g., microscope or CMOS chip). In some embodiments, a beam-split fraction of the pulsed excitation source, which may be used for example in a microscopy-based system, generates a delayed logic pulse relative to the timing of the excitation pulse/modulation going into the microscope. In the microscopy embodiment, the focused output light from the sample 3 impinging on the gated-intensifier 4 selects the light delayed relative to the excitation beam and therefore only the delayed light is focused and imaged by the imaging detector 5 (e.g., by a CCD camera).

In a chip-base exemplary embodiment, the detector gate 2, the implementer of the delay 4, and detector 5 are incorporated into an integrated semiconductor device (e.g., CMOS chip) and may be configured to prevent the fluorescent signal from the acceptor from being detected by the detector 5 or may allow the fluorescent signal from the acceptor to be detected by the detector 5 by timing via the detection capability of the pixels of the CMOS detector (i.e., turning the pixels on/off based on the timing scheme received from the delay logic at 2).

Accordingly, after the delay period Δt (which in an exemplary embodiment when using quantum dots as donors may be at least from about 10 ns to about 15 ns and can be longer in other FRET-based dye systems) from the excitation source emission, the gate 2 is timed to permit the fluorescent signal from the FRET-emitting acceptor dye to be detected by the detector. As noted above, the detector 5 can be various components, including but not limited to for example, a CCD device, a CMOS device, a detector array, a combination thereof, or the like. The detector may alternately be incorporated into a semiconductor device with the detector gate and may permit detection of the fluorescent signal through the semiconductor device. As will be discussed further, the detector gate 2 may, after reaching a second predetermined time (e.g., approximately 50 ns when using a quantum dot donor), be operated to prevent the subsequent detection of long lifetime autofluorescence, as some impurities may have longer fluorescence lifetimes than the lifetime of the quantum dot or fluorophore. The detector 5 in an exemplary embodiment may be a high density array of incorporation detectors integrated on the semiconductor substrate.

If the number of photons accumulated is not enough for the detector 5 to produce a meaningful signal-to-noise ratio of the image, as noted above, the system may include a gating image intensifier 4 utilized as part of the detector gate mechanism, such as, for example, a microchannel plate, operatively connected with the detector gate 2. For example, focused output light from a microscope unit impinging on the gating image intensifier 4 selects for the light delayed by the delay logic 2 relative to the excitation beam. The intensifier 4 thereby acts as an amplifier of the emitted photons. In some embodiments, gains of higher than 10⁴ can be achieved, and so the aggregate number of photons can be increased in some embodiments. Thus, not only can noise be decreased by timing the gate 2 to block and subsequently allow the detector 5 to detect the emitted fluorescent signal, but the signal can also be increased by the intensifier 4 or the gate/intensifier, giving an even larger signal-to-noise ratio for sequencing. In alternative embodiments, as mentioned above, the detector gate electron multiplying, electron bombardment, low noise CMOS detectors, or any other high-signal sensitivity detection may be utilized.

The excitation source 1 is pulsed in order to determine the timing of when the detector gate 2 is triggered to permit or block detection of the fluorescent signals at the detector 5. In some embodiments, the pulsed excitation source can exhibit a rise time of 10 ns or less, for example, 6 ns or less, 4 ns or less, or 2 ns or less. In some embodiments, the pulsed excitation source can exhibit a decay time of 25 ns or less, for example, 15 ns or less, 10 ns or less, or 7 ns or less. An exemplary gate that can be used is a 9MCP gate, available from Stanfordcomputeroptics.com. The intensifier gate 2 can be integrated or free standing with respect to the detector 5 and is used to “gate” the emission after the laser pulse in time, i.e. a few nanoseconds after the start of the laser pulse on the sample. The system can be configured to keep the gate open for many tens of nanoseconds or more, for example, as appropriate based on the types of FRET donors that are utilized in order to integrate as many photons as possible that have been emitted by the acceptor of the FRET-based dye system. When the gate 2 is incorporated into the semiconductor device, the gate 2 may be an electric charge that permits the fluorescent signals to be detected by the CMOS detector or blocks the fluorescent signals from being detected, or alternatively, the gate may be incorporated into the detector 4 and may cause the “gating” of the detector timing detection operation with the timing of the pulsed emission. In some embodiments, the gate can be used to monitor the FRET donor in a similar fashion.

Autofluorescence from objectives or other optical elements can be reduced according to the present teachings. The autofluorescence due to some impurities in the objective or other optical elements may have fluorescence lifetimes, which are longer, potentially much longer than the lifetime of the quantum dot (or other donor) or fluorophore, potentially many microseconds as described in EP 0492577, which is incorporated herein in its entirety by reference. Accordingly, detector gates in accordance with the present teachings may be configured to turn off again after the acceptor signal has been collected, prior to the emission of long lifetime autofluorescence.

According to various embodiments, the process is repeated in high repetition rates, for example, on the order of 0.5 MHZ or higher, 1.0 MHz or higher, or 5 MHz or higher. In some embodiments, modules are used with repetition rates of 200 kHz and the duty cycle of the gate integrating is high enough to collect enough photons for detection. In some embodiments, spatial uniformity and resolution implications of the intensifier are addressed in systems comprising a cathode and multichannel plate (MCP) or image intensifier, which can reduce the resolution of the combined detector if the modulation transfer function of the MCP or image intensifier results in the MCP or image intensifier having a limiting resolution which is worse than the pixilation of the image detector 5 (e.g., CCD device). Optical considerations, for example, increasing the size of the emission before the MCP or image intensifier, and then reducing it back to the face of the CCD, EMCCD, EBCCD or CMOS detector, can be used to minimize such spatial resolution issues.

According to various embodiments, a geometrical arrangement is provided comprising multiple detectors for the emitter/donor signal, with one gate in front of each detector. In an alternative embodiment, a diffractive element may be utilized prior to imaging on the multichannel plate or image intensifier; the resulting amplified image may be re-imaged onto the detector. Other configurations are also within the present teachings.

According to some embodiments, a single gate can be used in front of a spectral decomposition optical system. Using a gate that creates photons via a photocathode can result in the loss of spectral information, so the emission photons from each emitter can be separated spectrally directed to spatially different portions of the gate/intensifier. In such an embodiment, the respective portions of the gate/intensifier can each gate and intensify a different respective color. After the gate/intensifier or a gating/intensifying module, the separate colors can then be recombined appropriately to match a geometrical arrangement of the detector, for example, or a plurality of CCD cameras. In some embodiments, three emitters can be used on one camera, and one emitter and donor can be used on another camera. In some embodiments, like arrangements can be provided. In an alternative embodiment, an optoacoustic element may be used as a gate; such an element does not affect the spectral content of the emission beam. Accordingly, standard filters and dichroics may be utilized to spectrally separate different emission and or donor channels.

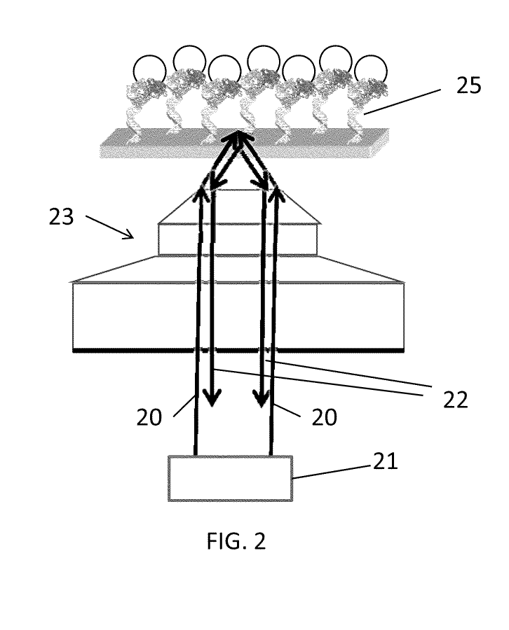

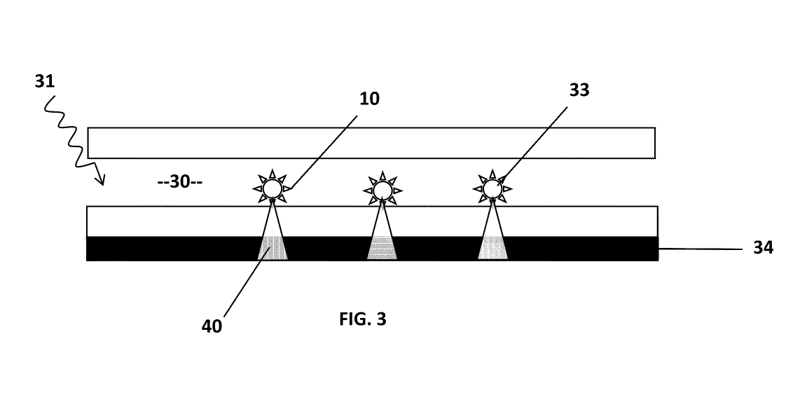

As mentioned above, the overall time-gated detection scheme illustrated schematically in FIG. 1 can be implemented using various system configurations. FIGS. 2 and 3 respectively depict exemplary embodiments of a microscope-based system and a semiconductor chip-based system which can implement a FRET-based dye system in combination with the time-gated fluorescence lifetime imaging (FLIM) detection in accordance with the present teachings in order to achieve detection/sequencing of single molecules of nucleic acids.

FIG. 2 is an exemplary embodiment of a microscope-based FRET/FLIM nucleic acid detection system that can be configured via appropriate circuitry and program controls to operate according to FIG. 1 described above when implemented using the microscope-based approach. A pulsed excitation beam 20 is transmitted from a pulsed or modulated excitation source 21, for example, to an optical microscope 23, which is focused on reaction sites 25 at which the nucleic acid molecule to be detected is located. An emission signal 22 emitted from respective reaction sites 25 is output from the microscope 23, and the detection of the emission signal 22 is delayed relative to transmission of the excitation beam 20. Specifically, in the manner described above with reference to FIG. 1, only the delayed light is focused and imaged by an imaging detector (not shown), such as, for example, a CCD or CMOS camera. Additional optics (not shown) also may be included to filter or enhance the emitted signal 22, including but not limited to, for example, a microchannel plate or other intensifier.

FIG. 3 is an exemplary embodiment of the semiconductor chip-based FRET/FLIM nucleic acid detection system that can be configured via appropriate circuitry and program controls to operate according to FIG. 1 described above when implemented using the chip-based approach. In the system, an excitation source 31 provides an excitation beam to a plurality of reaction sites 33 in a sample chamber 30 that is integrated with a semiconductor (e.g., CMOS) detector chip 34. Fluorescent signals 40 emitted from respective reaction sites 33, ultimately are detected by the detector 34 after a time delay from the initial transmission of the excitation beam 31. Delay logic (e.g., 2 in FIG. 1) is incorporated via appropriate circuitry into the semiconductor device and may operate in concert with the detector 34 to delay (prevent) detection or allow detection by the detector 34 of the fluorescent signal and ultimately provide a voltage readout of the detected signal to determine a base call. As with the embodiment of FIG. 1, other components may be included to enhance and/or filter the emitted signals 40 for detection.

In various exemplary embodiments, the excitation sources can be pulsed or modulated excitation sources, and may include, but are not limited to, for example, pulsed lasers, pulsed LEDs, pulsed solid state laser diodes, pulsed microwire lasers, etc.

According to various embodiments, the lifetimes, and fluorescence intensities over those lifetimes, of a number of different quantum dots, is shown in FIG. 4. An example of an acceptor-fluorescence, shown as the dotted line, can be seen to take on the effective lifetime of a donor-emission, as shown by the line denoted with squares (▪), generated by quantum dots having 7.3 dyes-per-Qdot-nanocrystal. Experiments performed with rod-shaped 605 nm Qdot-nanocrystals having no acceptor moiety exhibited a fluorescence lifetime of about 23 nanoseconds (ns). In some embodiments, spherical quantum dots can be used for single molecule sequencing and exhibit a measured lifetime of about 50 ns.

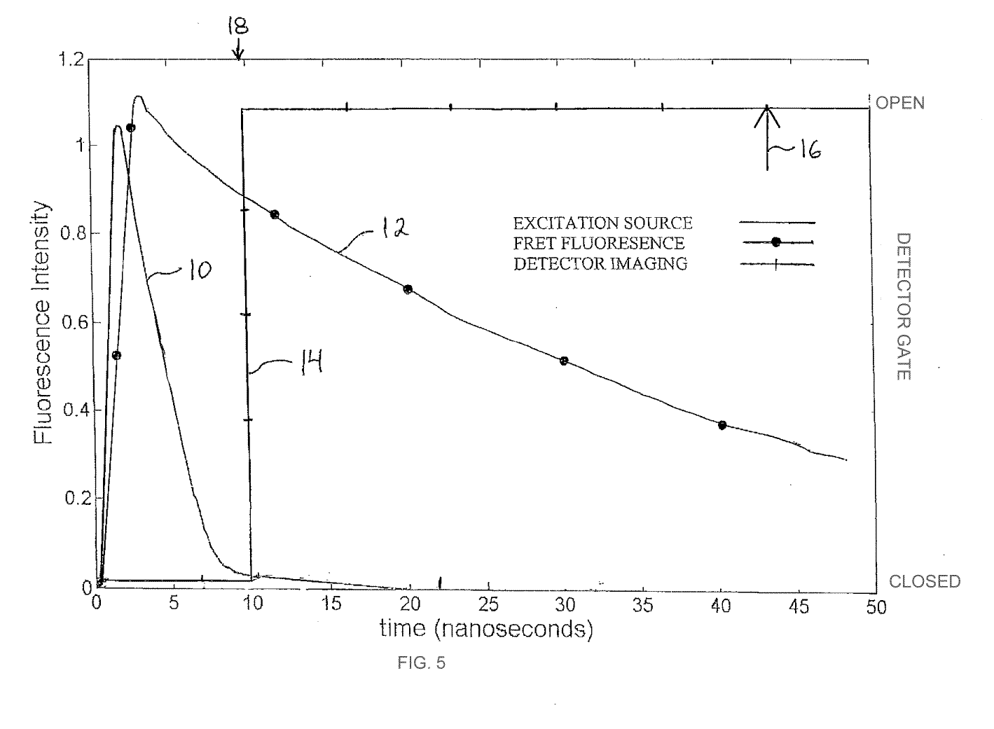

In the system used to generate the graph shown in FIG. 5, the excitation source emitted an excitation wavelength of 405 nm, and detection was done using a Photon Technology Instruments time-resolved instrument. The data fitted-lines were generated using Global Analysis with iterative convolution. An optimal laser repetition rate for the gating experiments was found to be about 5 times the donor lifetime, for example, 5 times a 50 ns lifetime, or 250 ns. The pulsing laser repetition rate was 1 per 250 ns, which is equivalent to a frequency of about 4 MHz, maximum. By adapting the system for 35% FRET, a frequency of about 6.25 MHz, maximum, can be used. In some embodiments, lower repetition rates can be used.

According to various embodiments, a detector is provided that implements a timing scheme as illustrated in FIG. 5. FIG. 5 is a graph showing a curve of the rise time and decay time of an excitation beam, a curve of the fluorescence decay of a quantum dot-based FRET dye system, and a curve showing the closed and open timing of a detector gate, in accordance with various embodiments of the present teachings. As can be seen, a fluorescence detector works in concert with or incorporates a detector gate that enables accumulation of signals from fluorescence emitted from a detection site, after a period of excitation from an excitation source. As can be seen, an excitation source is turned on for about 2 ns and the excitation beam causes autofluorescence of various components of the detection system (including fluorescence attributable to the substrate materials that define the detection site), for about 10 ns. Curve 10 shown in FIG. 5 demonstrates the detectable radiation that is directly attributable to the excitation source alone. During that 10 ns period of autofluorescence, the detector gate may be maintained in a closed configuration, or otherwise timed, to block detection of fluorescent signals at the detector such that detector elements of the detector, for example, pixels of a CCD camera or CMOS chip, are prevented from receiving the 10 ns autofluorescence signal.

The Y-axis illustrated on the right-hand side of the graph of FIG. 5 shows the two positions of the detector gate, open and closed. Curve 14 in FIG. 5 represents the configuration of the detector gate relative to the X-axis time line. As can be seen, at a point in time 18, which corresponds to about 10 ns along the time axis, the configuration of the detector gate may be opened, enabling the detector elements to receive and accumulate a fluorescence signal emitted from the reaction site. Point 16 along the time axis of curve 14 indicates a point at which an intensifier can be turned on, for example, to amplify or otherwise intensify the received fluorescence signal. An intensifier may be used to boost the ever-weakening fluorescence signal received by the detector.

Curve 12 in FIG. 5 shows the fluorescence intensity emitted from the reaction site and the decay of the intensity over time. The fluorescence represented by curve 12 is that generated by a quantum dot-based FRET dye system used according to various embodiments of the present teachings. As one skilled in the art will appreciate, many of the quantum dot-based dyes exemplified in FIG. 5 can be used in conjunction with such a detection system.

According to various embodiments, appropriate FRET dye systems can be selected or produced to meet exacting specifications. In some embodiments, a gated acceptor and donor detection system is provided for quantum dot nanocrystal-based single molecule sequencing applications. In an example, by comparison, non-FRETing dye dNTP's have a fluorescence lifetime of about 3 ns. An effective lifetime of a FRETing dye dNTP, however, can be calculated as a convolution of the lifetime of the donor moiety and the lifetime of the acceptor moiety. The calculations can yield results that show quantum dot nanocrystals of the SDot-type having a donor lifetime of about 50 ns.

In systems and methods of the present teachings using quantum dot nanocrystals of the SDot-type, the measured FRETing acceptors can be “delayed” or spatially separated from the prompt emission signal of non-FRETing background dNTP's, at a ratio of Excitation Source Time (times) non-FRETing donor lifetime that comes to about 15 ns. Thus, it can be calculated that in 15 ns, 99.4% of the signal generated from the non-FRETing donor, has been emitted, yet much of the emission signal from a quantum dot-based FRETing dye as used herein, would still be generated after 15 ns.

By implementing an exemplary gated detection system as described herein, approximately 99% of the dye-dNTP non-FRETing background can be removed while sacrificing only about 25% of the acceptor signal from the quantum dot-based FRETing acceptors used according to the present teachings.

According to various embodiments, the gate-on-time can be adjusted back-and-forth dependent on whether there is a particular photon species that is more desired to detect. For example, a 9 ns gate yields a donor background decrease of about 95% and an acceptor decrease of only 17%. Moreover, such a gating scheme also eliminates essentially all optical scattering and solvent RAMAN scattering.

FIG. 6 is a table showing the fluorescent lifetime of various exemplary dyes, which may be used as part of a FRET-based dye system in accordance with various exemplary embodiments of the present teachings.

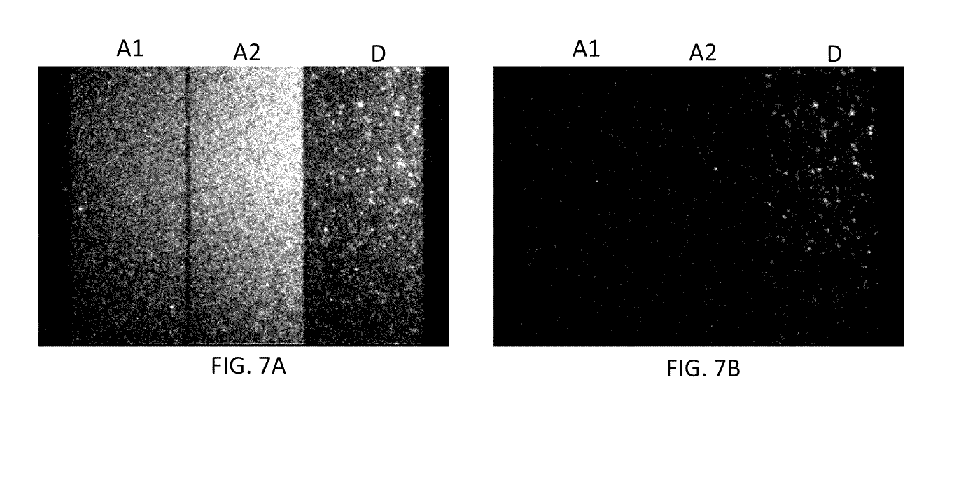

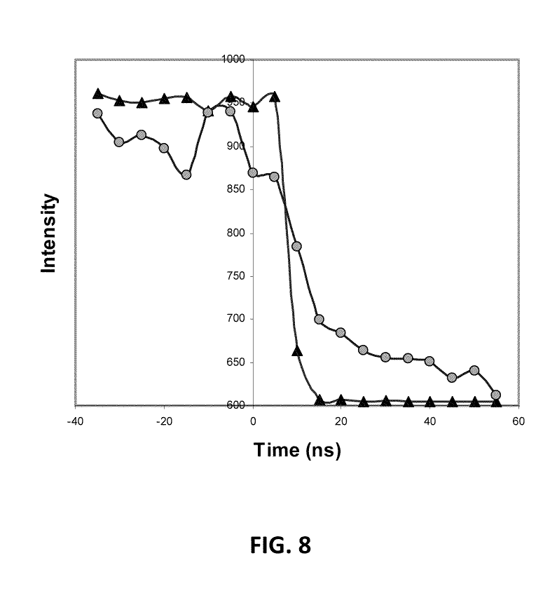

With reference now to FIGS. 7 and 8, comparative data obtained from a single molecule sequencing reaction with and without the use of a time-gated FLIM technique in accordance with the present teachings are shown. FIGS. 7A and 7B respectively show images obtained of fluorescent emissions at a donor (D) and two acceptor (A1, A2) fluorescent emission channels without a time-gated detection system (FIG. 7A) and with a time-gated detection system (FIG. 7B) in accordance with various embodiments of the present teachings. FIG. 7A shows CCD camera images of single molecule quantum-dot nanocrystal sequencers bound to primer-template DNA as observed at three different fluorescence emission channels: donor channel (D) centered at 605 nm (+/−˜10 nm), along with two acceptor detection channels (A1, A2) centered at 660 nm (+/−20 nm) and 720 nm (+/−20 nm) respectively, without time-gating of the camera. In the absence of any time-gating (no time-gating case), the fluorescent images obtained in the A1 and A2 channels shown in FIG. 7A revealed a large amount of prompt fluorescence emission from the dye, as depicted by the white areas in the images.

FIG. 7B shows CCD camera images of single molecule quantum-dot nanocrystal sequencers bound to primer-template DNA as observed at three different fluorescence emission channels: donor channel (D) centered at 605 nm (+/−˜10 nm), along with two acceptor detection channels (A1, A2) centered at 660 nm (+/−20 nm) and 720 nm (+/−20 nm) respectively, with time-gating of the camera in accordance with at least one embodiment of the present teachings. To obtain the images shown in FIGS. 7A and 7B, the excitation source was a 405 nm laser (Picoquant), at 1 MHz repetition rate and 67 uW of average power. A solution of Alexa-Fluor 647 dye-labeled deoxynucleotides (˜10 uM) filled a flow chamber above the individual quantum-dot sequencers. An intensified Princeton Instruments PI-Max 3 CCD camera was utilized. The camera supplied the logic to control the timing between the laser pulse and the gate on the camera's image intensifier (“master-mode” operation). In the presence of a 5 ns delay and a 250 ns on-gate, the fluorescence image (with time-gating) was obtained (see FIG. 7B). After waiting approximately 5 ns for all of the prompt Alexa Fluor 647 emission to decay, the imaging of the long-lived fluorescence from the quantum dot sequencers is clearly visible without the additional fluorescence from the Alexa Fluor 647 emission (i.e., the white areas and fewer are more discrete) due to the delayed time because of the long emission lifetime of the quantum-dot nanocrystal material.

FIG. 8 is a graph showing average intensities of fluorescent emissions as a function of the time delay between the laser pulse input and the turning on of the image intensifier. The intensities are depicted for the acceptor A1 channel (triangles) and a donor D channel (circles) corresponding to the time-gated images like FIG. 7B. The time-delay was scanned progressively in 5 ns intervals and was on for 250 ns. Time t=0 corresponds to the rising edge of the laser pulse exciting the sample. As depicted in FIG. 8, as the time-delay increases after the laser pulse, the signal intensity observed in the acceptor channel (A1) decreased much more rapidly than in the donor channel (D) due to the longer fluorescent lifetime of the quantum-dot sequencer than the Alexa Fluor 647 labeled deoxynucleotide. By ˜15 ns after the laser pulse, all of the A1 channel emission was eliminated down to the read-noise of the camera (˜600 units), while the quantum-dot sequencing signal was still clearly visible. The signals represent the accumulation of 60,000 individual laser pulses.

While it has been discussed to terminally label the four nucleotides with four different color dyes and provide the four nucleotides to the nucleic acid molecule being detected/sequenced, each of the four nucleotides may be terminally labeled with FRET acceptor dyes and may be provided to the reaction site one nucleotide one color at a time, rather than at the same time, without departing from the scope of the present teachings.

Further, although many of the above embodiments are described as using a single molecule sequencing reaction that uses nucleotide incorporation reactions, it is contemplated as being within the scope of the present teachings that the time-gated fluorescent lifetime imaging systems and method described herein can be used in conjunction with sequencing reactions that utilize polymerase-dependent nucleotide transient-binding reactions, as disclosed, for example, in International Publication WO/2010/141390, entitled “Nucleotide Transient Binding for Sequencing Methods,” incorporated by reference herein in its entirety.

Other excitation sources, detectors, electronics, processors, components, methods, and the like, that can be used according to the present teachings include those described, for example, in U.S. Published Patent Application No. US 2009/0146076 A1 to Chiou et al., in Poher et al., Video Rate Fluorescence Lifetime Imaging and Structured Illumination Using a Blue LED, Imaging Sciences Centre, Photonics Group, Department of Physics, Imperial College London, and in the publication from crackerbio entitled Description of our technology, cracker, from cracker[@] ITRI, www.crackerbio.com, each of which is incorporated herein in its entirety by reference.

Further modifications and alternative embodiments will be apparent to those skilled in the art in view of the disclosure herein. For example, the systems and the method may include additional components or steps that were omitted from the diagrams for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

Those having skill in the art would recognize that the various exemplary embodiments described herein may be modified to perform a variety of assays, and although some specific examples for which the systems and methods may be well-suited are disclosed, such examples are nonlimiting and exemplary only. By way of example, various time delays, excitation frequencies and spectra, and repetition rates are disclosed, which may be suitable for use in conjunction with various exemplary embodiments. However, based on the present teachings, those having ordinary skill in the art would understand how to select such parameters depending for example, on the FRET components selected (e.g., donor types and acceptor types) and/or other factors, in order to carry out the time gated fluorescence lifetime imaging methods and systems taught herein.

Those having ordinary skill in the art would understand that features, components, steps, and/or materials described with respect to a particular exemplary embodiment set forth herein may be used with one or more other exemplary embodiments set forth herein and modifications made accordingly. It is to be understood that the particular examples and embodiments set forth herein are nonlimiting, and modifications to structure, dimensions, materials, and methodologies may be made without departing from the scope of the present teachings.

Other embodiments will be apparent to those skilled in the art from consideration of the specification and practice of the present teachings disclosed herein. It is intended that the specification and examples be considered as exemplary only, with a scope being of a breadth indicated by the claims, including their full scope of equivalents.

What is claimed:

1. A nucleic acid detection system, comprising:
a pulsed excitation source transmitting excitation energy to a reaction site including a fluorescence resonance energy transfer (FRET)-based dye system to generate a fluorescent signal at the reaction site;
a detector configured to detect the fluorescent signal from the reaction site; and
a detector gate configured to respectively block and permit detection of the fluorescent signal at the detector, said detector gate being timed based on an emission start time of the transmitted excitation energy.

2. The detection system according to claim 1, wherein the FRET-based dye system comprises at least one of a quantum dot, a lanthanide, a ruthenium, and an acridine as a donor in the FRET-based dye system.

3. The detection system according to claim 2, wherein the at least one quantum dots comprises a quantum dot nanocrystal of the S-dot-type.

4. The detection system according to claim 2, wherein the FRET-based dye system comprises differing organic dyes labeling each of four nucleotides to be incorporated as acceptors in the FRET-based dye system.

5. The detection system according to claim 1, wherein the detector comprises one or more charge-coupled device (CCD) cameras.

6. The detection system according to claim 1, wherein the detector gate further comprises an intensifier to amplify the fluorescent signal detected by the detector.

7. The detection system according to claim 1, further comprising:
electrical circuitry and logic synchronizing the pulsed excitation source and the detector gate.

8. The detection system according to claim 7, wherein the detector gate blocks detection of the fluorescent signal by the detector for a first predetermined amount of time from the emission start time.

9. The detection system according to claim 8, wherein the first predetermined amount of time is about 10 nanoseconds.

10. The detection system according to claim 8, wherein the detector gate permits detection of the fluorescent signal by the detector after the first predetermined amount of time has elapsed.

11. The detection system according to claim 10, wherein the detector gate blocks detection of the fluorescent signal after a second predetermined amount of time from the emission start time has elapsed.

12. The detection system according to claim 11, wherein the second predetermined amount of time is about 50 nanoseconds.

13. The detection system of claim 1, wherein the pulsed excitation source comprises a pulsed laser.

14. The detection system of claim 1, wherein the pulsed excitation source comprises a modulated laser.

15. A method of detecting a nucleic acid molecule sequence, comprising:
reacting at a reaction site a nucleic acid molecule with a fluorescence resonance energy transfer (FRET)-based dye system;
turning on an excitation source and transmitting excitation energy to the FRET-based dye system to generate fluorescent emissions at the reaction site;
preventing detection, by a detector gate, of the fluorescent emissions at a detector after an emission start time of the transmitted excitation energy;
turning off the excitation source;
permitting detection of the fluorescent emissions after a first predetermined amount of time from the emission start time has elapsed;
detecting the fluorescent emissions with the detector to form a detected signal; and
determining a character or sequence of the DNA molecule based on the detected signal.

16. The method according to claim 15, further comprising additionally blocking detection, by the detector gate, of the fluorescent emissions at the detector after a second predetermined amount of time from the emission start time has elapsed.

17. The method according to claim 15, wherein a FRET donor of the FRET-based dye system has a fluorescence decay lifetime of at least 10 nanoseconds

18. The method according to claim 15, wherein the excitation source comprises a pulsed laser source and the method comprises repeatedly pulsing the pulsed laser source between an on configuration in which the pulsed laser source generates excitation energy, and an off configuration in which the pulsed laser source is turned off and does not generate excitation energy.

19. The method according to claim 15, wherein the determining a character or sequence of the nucleic acid molecule based on the detected signal comprises sequencing the nucleic acid molecule.

20. The method according to claim 15, further comprising intensifying the fluorescent emissions detected with the detector.

21. A method of detecting a nucleic acid molecule sequence, comprising:
reacting at a reaction site a nucleic acid molecule with a fluorescence resonance energy transfer (FRET)-based dye system;
transmitting excitation energy to the reaction site to excite the FRET-based dye system to generate fluorescent emissions at the reaction site; and
syncing timing of the excitation energy transmission with detection of the fluorescent emissions by a detector and detecting the fluorescent emissions at the detector after a predetermined delay time from an emission start time of the transmitted excitation energy.

22. The method according to claim 21, further comprising preventing detection of the fluorescent emissions at the detector after the fluorescent emission start time and before the first predetermined delay time has elapsed.
